(19) United States
(12) Patent Application Publication
(10) Pub. No.: US 2024/0261203 A1
(43) Pub. Date: Aug. 8, 2024

(54) ALCOHOL-FREE NANOEMULSION PERFUME

(71) Applicant: LVMH RECHERCHE, SAINT JEAN DE BRAYE (FR)

(72) Inventors: Solène GODARD, Saint-Jean-de-Braye (FR); Kenta MITANI, Chiyoda-Ku, Tokyo (JP); Mai OZAWA, Chiyoda-Ku, Tokyo (JP)

(21) Appl. No.: 18/568,007

(22) PCT Filed: Jun. 15, 2021
(86) PCT No.: PCT/EP2021/066139

§ 371 Date: Dec. 7, 2023

(51) Int. Cl. A61K 8/55; A61K 8/06; A61K 8/37; A61Q 13/00

(52) U.S. Cl. CPC A61K 8/553; A61K 8/062; A61K 8/375; A61Q 13/00; A61K 2800/30

(57) ABSTRACT

The invention relates to an oil-in-water emulsified perfume cosmetic comprising less than 10% by mass of lower alkyl alcohol such as ethanol, a) at least 40% by mass of water, b) at least 6% by mass of a perfume concentrate, c) at least one hydrogenated lecithin, and d) at least one high gravity oil having a density at 20° C. that is at least 0.90 g/cm3.

### TECHNICAL FIELD

The present invention relates to a new perfuming cosmetic composition which can be an eau de toilette, an eau de Cologne, a perfume extract, a perfume spirit or a perfume water according to the content of perfume concentrate which it contains and the notes which it expresses. This perfuming composition has excellent properties such as the olfactory fidelity of the perfume concentrate and the remanence of the perfumed note over time after application, the absence of a sticky sensation to the touch, and the ability to be sprayed under the form of fine particles.

### BACKGROUND ART

Alcohol free perfumes find some interest for certain consumers who believe that alcohol may not be good for skin and that skin irritation may occur with other cosmetic products containing alcohol, and who would like to use perfume products. Thus, there is an unmet consumer need for alcohol free fragrance products.

Besides, alcohol such as ethanol plays an important role for perfume formulation thanks to its high ability to dissolve fragrance. Many approaches have been explored in the prior art in order to prepare suitable perfume cosmetics containing no alcohol. For example, as an alternative approach for using ethanol, nonionic surfactants have been proposed as a way to dissolve perfumes (WO 2017/059513 and CN 103637942). However, high quantities of nonionic surfactants are necessary to dissolve the perfume concentrate. Such high amounts impair sprayability due to an induced thixotropic fluidity. They also affect perfume olfactive quality and provide a harsh green and acid note. Furthermore, high amount of surfactants may lead to stability problems.

### Technical Problem

Surfactants are crucial components for non-alcohol perfume products to dissolve or emulsify fragrances, in particular in the case of perfume products that contain high quantity of fragrance such as 12% by mass. For concentrated perfume products that are prepared with a conventional emulsification process, it is necessary to dissolve high quantity of fragrance with high quantity of surfactants, typically more than 10% by mass and up to 25% by mass.

Common oil-in-water emulsified perfume cosmetics, in particular those which contain high amounts of perfume concentrates, have impaired emulsion stability and olfactive fidelity over time. They also tend to produce a liquid cosmetic that cannot be sprayed on skin or clothes.

Therefore, there is a need to propose non-alcohol perfume formula which provides high sprayability while maintaining stability and olfactive properties of perfume cosmetics.

### SUMMARY OF THE INVENTION

It is an object of the invention to provide oil-in-water emulsified perfume cosmetics that contain very low amounts of alcohol, or no alcohol at all, while exhibiting excellent stability and sprayability.

The present invention provides a gentle perfume formula without using alcohol. It is in the form of a nanoemulsion comprising high quantity of fragrance and has high sprayability while securing emulsion stability over time and olfactive stability over time. The formula does not advantageously contain high quantity of surfactant(s), in particular nonionic surfactants.

A goal of the present invention is to develop a perfume formula by dissolving sufficiently high percentages of fragrance, without alcohol and without high amount of surfactants. Such a formula enables high sprayability, while keeping the same quality and properties of alcohol based perfumes such as light sensorial finish, olfactive properties and long-term stability.

In the sense of the invention, “stability” can be observed in at least one of the following storage conditions: storage at 50° C. and 25° C. for one month, storage at −18° C. for one week; and storage with temperature variation cycles (for example a cycle at 40° C. for 12 hours and −10° C. for 12 hours, that is repeated for one month). According to one embodiment of the invention, stability can be observed after storage at 25° C. for one month, and/or storage at 50° C. for one month. Stability can be assessed at the naked eye, and may be considered as no creaming, and/or no phase separation between an oil phase and a water phase, and/or no coalescence of oil particles in the nanoemulsion.

“Sprayability” means that the perfume cosmetic can be sprayed in the form of fine droplets by means of pressurisation devices. These devices are well known to the skilled person and include non-aerosol pumps or “atomisers”, aerosol containers comprising a propellant as well as aerosol pumps using compressed air as propellant.

### Solution to Problem

The present invention solves the above related problems and disadvantages of the alcohol-free prior art perfume formula and provides an oil-in-water emulsified perfume cosmetic comprising less than 10% by mass of lower alkyl alcohol such as ethanol, at least one hydrogenated lecithin and one high gravity oil. The perfume cosmetic is preferably a nanoemulsion.

The oil-in-water emulsified perfume cosmetic of the invention can accommodate large amounts of perfumes while exhibiting excellent stability without large amounts of ethanol and surfactants required in the prior art. Moreover, it has an excellent sprayability, and the surfactant level is drastically reduced.

### DESCRIPTION OF EMBODIMENTS

The present invention relates to an oil-in-water emulsified perfume cosmetic comprising less than 10% by mass of lower alkyl alcohol such as ethanol, a) at least 40% of water, b) at least 6% of a perfume concentrate, c) at least one hydrogenated lecithin, and d) at least one high gravity oil having a density at 20° C. of at least 0.90 g/cm³, better at least 0.95 g/cm³, preferably that is from 0.95 g/cm³ to 1.10 g/cm³.

In the context of the invention, the expression “from . . . to” is intended to include the upper limit value and the lower limit value of the range, whereas the expression “between . . . and” is intended to exclude the limits of the range. In the expression “at least . . . x %”, the content is equal to x % or higher than x %.

In the present description, the percentages are expressed by mass with respect to the mass of the perfume cosmetic, unless defined otherwise.

“The oil-in-water emulsified perfume cosmetic” of the invention can be interchangeably worded in the present description as “the perfume cosmetic”, “the cosmetic”, “the perfume composition”, “the perfuming composition” or “the composition”.

### Emulsion

The cosmetic of the invention is an oil-in-water emulsion and preferably comprises an oil phase and a water phase, said oil phase being dispersed in the water phase. The average particle size of emulsion particles of the cosmetic of the invention is preferably from 10 nm to 500 nm.

Emulsion particles in the sense of the invention can be oil droplets that are dispersed in water. Such an average particle size range advantageously provides a stable perfume cosmetic.

The average particle size of the emulsion particles is preferably from 10 nm to 300 nm. According to the invention, “average particle size” means D50 particle size and can also me named as “average hydrodynamic diameter.

The average particle size of the emulsion particles can be measured using a particle size measuring device (DelsaMax® CORE: manufactured by Beckman Coulter, Inc.) according to cumulant analysis.

A high-pressure emulsifying apparatus can be used to prepare the cosmetic of the invention, by way of mixing (a) to (d) ingredients with other additives if present, in plural times at a high pressure. This high-pressure emulsifying process is one mean for setting the average particle of the emulsion particles within the above-mentioned range.

The cosmetic of the invention is preferably an oil-in-water nanoemulsion. An “oil-in-water nanoemulsion” according to the invention means an oil-in-water emulsion comprising oil globules having an average particle size being lower than than 500 nm, preferably from 10 nm to 500 nm, an still preferably from 10 nm to 300 nm. The small size of the oily globules is generally obtained by virtue of the use of mechanical energy and in particular of a high-pressure homogenizer.

The nanoemulsions are to be differentiated from microemulsions by their structure.

Indeed, microemulsions are thermodynamically stable dispersions consisting, for example, of micelles of amphiphilic lipid(s) swollen with oil. In addition, microemulsions do not require significant mechanical energy to be produced; they form spontaneously simply by bringing the constituents into contact.

### Ethanol Content

The cosmetic of the present invention contains less than 10% by mass of at least one lower alkyl alcohol. A lower alkyl alcohol can be an aliphatic monoalcohol comprising 2 to 6 carbon atoms. Such a lower alkyl alcohol can be ethanol.

The lower alkyl alcohol content, in particular the ethanol content, is less than 10.0%, preferably less than 5.0%, and even more preferably less than 2.0%.

The composition is preferably free of any lower alkyl alcohol. In a particular embodiment, the cosmetic may be free of ethanol, or may contain less than 1.0%, preferably less than 0.1% ethanol.

In the meaning of the present invention, “free of ethanol” or “alcohol-free” can also designate a cosmetic that is prepared by a process for preparing that preferably comprise no step of adding a lower alkyl alcohol and no step of mixing a lower alkyl alcohol with other ingredients. It cannot be excluded that traces of lower alkyl alcohol, in particular traces of ethanol, can be present in some of the ingredients that are used to prepare the cosmetic of the invention. In such a case the cosmetic of the invention may still be named as an “alcohol free oil-in-water emulsified perfume cosmetic”.

The cosmetic of the invention may be an alcohol free oil-in-water emulsified perfume cosmetic, in particular a lower alkyl alcohol free oil-in-water emulsified perfume cosmetic or an ethanol free oil-in-water emulsified perfume cosmetic.

### Water

The composition according to the invention comprises a) at least 40%, preferably at least 50% water, preferably at least 55% of water, and more preferably at least 60%.

The water content in the composition may be higher than a minimum value equal to 50%, 55%, 60% or 65%. The water content in the composition may be lower than a maximum value equal to 70%, 75% or 80%. The water content ranges, for example, from 55% to 80%, preferably from 60% to 70%.

Water can be selected in the group consisting of steam-distilled water that is derived from plants, distilled water, purified water, hot spring water, deep water, and mixtures thereof. A steam-distilled water that is derived from plants can be lavender water, rose water or orange flower water.

The water phase of the oil-in-water emulsified perfume cosmetic of the invention may consist of water alone or may comprise water and water-soluble components.

### Perfume Concentrate

The composition contains b) a perfume concentrate. The perfume concentrate can be for example chosen from compounds having “Perfume” or “Fragrance” as an INCI name. A perfume concentrate is a compound or a mixture of compounds which is at least partially volatile at room temperature and which odor can be detected. A perfume concentrate made up of essential oils is generally diluted in order to express its full olfactory potential, i.e. a perception that evolves over day after application to the skin, thanks to the presence of several odorous organic compounds having volatilities that are different from one another. The elaboration of a perfume concentrate includes a step of associating several perfuming raw materials to give a top note, a middle note and a bottom note to the perfume composition.

The perfume concentrate can be prepared from natural or synthetic organic perfume materials.

Examples of naturally occurring fragrance materials are extracts of flowers, stems and leaves, fruits, fruit bark, roots, woods, herbs and grasses, resins and balsams. These vegetable fragrance materials may be essential oils, such as bergamot, rose, lavender, sandalwood, cardamom, sage, chamomile, clove, lemon balm, mint, cinnamon leaf, juniper, vetiver, olibanum, galbanum and labdanum.

Examples of synthetically derived perfuming materials are hedione, ethylene brassilate and habanolide, benzyl acetate, benzyl benzoate, phenoxyethyl isobutyrate, p-tert-butylcyclohexyl acetate, citronellyl acetate, citronellyl formate, geranyl acetate, linalyl acetate, dimethylbenzylcarbinyl acetate, phenylethyl acetate, linalyl benzoate, benzyl formate, ethylmethylphenyl glycinate alkylcyclohexyl propionate, styralyl propionate and benzyl salicylate, benzylethylether, linear alkanals with 8 to 18 carbon atoms, citral, citronellal, citronellyloxyacetaldehyde, cyclamenaldehyde, hydroxycitronellal, ionones such as alpha-isomethylionone, and methylcedrylketone, anethol, citronellol, eugenol, isoeugenol, geraniol, linalool, phenylethyl alcohol, terpineol, terpenes. These compounds often occur as a mixture of two or more of these odorants.

The perfume concentrate represents at least 6%, at least 7% or at least 8%, preferably at least 10%.

The amount of the perfume concentrate may range from 6% to 40%, preferably from 8% to 30% or more preferably from 10% to 30%. In an embodiment, the perfume concentrate represents from 10% to 15%.

### Hydrogenated Lecithin

The perfume cosmetic of the invention comprises c) at least one hydrogenated lecithin.

The hydrogenated lecithin amount preferably ranges from 0.1% to 5.0%, preferably from 0.2% to 3.0%, and still preferably from 0.3% to 1.0%.

Hydrogenated lecithin is a phospholipid composed of a phosphatidylcholine group and two fatty acids. One example of hydrogenated lecithin comprises two saturated fatty acid moieties having 16 to 18 carbon atoms.

The perfume cosmetic may further comprise at least one sterol, preferably at least one phytosterol, preferably at least one soybean sterol.

According to one embodiment of the invention, the perfume cosmetic comprises a mixture of at least one hydrogenated lecithin and at least one phytosterol. The perfume cosmetic can be prepared with one mixture of hydrogenated lecithin and phytosterols that is available on the market and sold under the trademark PHYTOCOMPO® by Nippon Fine Chemicals.

### High Gravity Oil

The perfume cosmetic of the invention comprises d) at least one high gravity oil having a density at 20° C. of at least 0.90 g/cm³, preferably at least 0.95 g/cm³, that is from 0.95 g/cm³ to 1.10 g/cm³. The density at 20° C. can be for example from 1.00 g/cm³ to 1.05 g/cm³. The high gravity oil is preferably liquid at 20° C. The density of the high gravity oil can be measured by any method known from one skilled in the art.

The high gravity oil can be selected from crosslinked tryglycerides, for example triglycerides obtained from caprylic acid and capric acid that are crosslinked with an aliphatic dicarboxylic acid having from 3 to 6 carbon atoms. An example of such a dicarboxylic acid is succinic acid.

The high gravity oil can be a caprylic/capric/succinic triglyceride oil, can have the INCI name “CAPRYLIC/CAPRIC/SUCCINIC TRIGLYCERIDE”, or can be an oil purchased on the market such as MYGLYOL® 829, which is a trademark from IOI Oleo. MYGLYOL® 829 is obtained from caprylic/capric glyceride units that are crosslinked with succinic acid.

The high gravity oil can also be an oil having the INCI name “Bis-Ethoxydiglycol Cyclohexane 1,4-Dicarboxylate”, and can be purchased as Neosolue™-Aqulio produced by Nippon Fine Chemicals. Such an oil can have a gravity being 1.08 g/cm³. The at least one high gravity oil amount preferably ranges from 0.1% to 5.0%, preferably from 0.2% to 3.0%, and still preferably from 0.5% to 2.0%.

### Block Copolymer of Alkyleneglycols

The cosmetic of the invention may further comprise at least one block-copolymer of ethylene glycol and propylene glycol, also known as polycondensate of ethylene glycol and propylene glycol, such as, for example, polyethylene glycol/polypropylene glycol/polyethylene glycol triblock block-copolymers (polycondensates). Such a copolymer can have the INCI name “POLOXAMER”.

These triblock polycondensates have, for example, the following chemical structure:

H—(O—CH₂—CH₂)ₐ—(O—CH(CH₃)—CH₂)_{b}—(O—CH₂—CH₂)ₐ—OH

in which formula “a” ranges from 2 to 150 and “b” ranges from 1 to 100; preferably, “a” ranges from 10 to 130 and “b” ranges from 20 to 80.

The block-copolymer (polycondensate) of ethylene oxide and of propylene oxide preferably has a mass-average molecular mass ranging from 1.000 to 20.000, better still ranging from 1.500 to 19.000, in particular ranging from 2.000 to 18.000 and even better still ranging from 4.000 to 17.000.

Mention may be made, as block-copolymer (polycondensate) of ethylene oxide and of propylene oxide which can be used according to the present invention, of the polyethylene glycol/polypropylene glycol/polyethylene glycol triblock polycondensates sold under the “Synperonic” names, such as “Synperonic® PE/F32” (INCI name: Poloxamer 108), “Synperonic® PE/F108” (INCI name: Poloxamer 338), “Synperonic® PE/L44” (INCI name: Poloxamer 124), “Synperonic® PE/F127” (INCI name: Poloxamer 407), “Synperonic® PE/F88” (INCI name: Poloxamer 238) or “Synperonic® PE/L64” (INCI name: Poloxamer 184 by Croda or also “Lutrol®68” (INCI name: Poloxamer 188), sold by BASF.

The copolymer of ethylene glycol and propylene glycol amount can be from 0.05% to 1%, preferably from 0.1% to 0.5%, and still preferably from 0.1% to 0.3%.

### Polyol

The cosmetic of the invention may comprise at least one polyol. One polyol can have from 2 to 6 carbon atoms and from 2 to 4 hydroxyl groups. Examples of such a polyol include ethylene glycol, 1,3-propanediol, polypropylene glycol, butylene glycol, pentylene glycol, hexylene glycol, dipropylene glycol, glycerin and diglycerin. Among these, butylene glycol, pentylene glycol, glycerin, and a combination of the foregoing are preferred.

One polyol, two polyols or more than two polyols can be used in the cosmetic. A mixture of three polyols being a mixture of glycerin, pentylene glycol and butylene glycol is used in a particular embodiment.

The content of the polyol can be from 5.0% to 25.0%, preferably from 10.0% to 20.0%, and still preferably from 15.0% to 20.0%.

The cosmetic of the invention may comprise from 10.0% to 20.0% of at least one polyol comprising from 2 to 6 carbon atoms and from 2 to 4 hydroxyl groups Advantageously, the composition of the invention consists essentially of water, perfume concentrate, hydrogenated lecithin, high gravity oil and polyol(s). By “essentially consisting” is meant that the sum of the percentages of water, perfume concentrate and polyol(s) is greater than or equal to 95%, or even greater than or equal to 98%.

### Additives

In addition to the ingredients described above, the composition may contain at least one cosmetically acceptable additive selected from dyes, preservatives other than glycols, buffers, electrolytes, UV filters, and anti-oxidants.

These additives should preferably be olfactively neutral so as not to alter the odor of the perfume composition, should not cause a change in color, and should not cause stability problems.

The dyes are for example: Caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, Cochineal carmine (CI 15850, CI 75470), Ext. Violet 2, Red 6-7, Ferric Ferrocyanide, Ultramarines, Acid Yellow 3/Yellow 10, Acid Blue 3, Yellow 10. Examples of fat-soluble dyes are Sudan Red, D&C Red 17, D&C Green 6, Beta Carotene, Soybean Oil, Sudan Brown, D&C Yellow 11, D&C Violet 2, D&C Orange 5, Quinoline Yellow, Annatto. The dyes generally represent from 0.01% to 1.0%, preferably from 0.05% to 0.5%.

The water phase of the oil-in-water emulsified perfume cosmetic of the invention may consist of water alone or may comprise water and water-soluble components. Some of these water-soluble components can be additives as described above.

The perfume cosmetic is preferably free of any anionic surfactant.

In one embodiment, the perfume cosmetic composition is preferably free of additional surfactant other than lecithin and alkylene glycol copolymer.

### Emulsification Process

The invention also describes a process for preparing the oil-in-water emulsion as described above.

The oil-in-water emulsified perfume cosmetic can be obtained by a step of emulsifying a mixture components (a) to (d) in a high-pressure homogenizer at a pressure being 100 MPa or higher.

The emulsification is preferably conducted at a pressure of 100 MPa or higher, the pressure preferably being 150 MPa or higher and more preferably 175 MPa or higher, and preferably being no higher than 225 MPa. The emulsification can be carried out using a high-pressure homogenizer (e.g., Star Burst® manufactured by Sugino Machine Limited) that carries out emulsification by pressurizing a fluid with a pump and ejecting it through fine slits provided in the flow channel.

The process of the invention may comprise a step of preparing a water phase comprising water, and any water-soluble additive if present, at a temperature that is between 20° C. and 60° C., a step of preparing an oil phase comprising the perfume concentrate and the hydrogenated lecithin, at a temperature that is between 20° C. and 60° C., a step of mixing the water phase and the oil phase at a temperature that is between 20° C. and 60° C. and with stirring at a revolution speed higher than 10,000 rpm, a step of allowing the mixture to cool down to 25° C. with further stirring at a revolution speed higher than 10,000 rpm, and a step of emulsifying the mixture in a high-pressure homogenizer at a pressure that is higher than 100 MPa.

The perfume cosmetic according to the present invention can be produced according to a process.

In a first step of the process, water and any other water-soluble additive if present, are heated at a temperature T, mixed and stirred (for example at a speed of 2500 rpm) using a stirrer so as to be wholly homogeneous, in order to obtain a water phase. The temperature T is preferably higher than 30° C., still preferably higher than 40° C.

In a second step, the perfume concentrate, the high gravity oil, and the hydrogenated lecithin, and any other oil soluble additive if present are heated at a temperature that is preferably equal to T, mixed and stirred under the same conditions as in the first step, so as to be wholly homogeneous, in order to prepare an oil phase.

In a third step, the water phase obtained in the first step, and the oil phase obtained in the second step are stirred with a homogenizer, and the mixture is cooled from temperature T to room temperature.

In a fourth step, the cooled mixture obtained in the third step is emulsified at a high pressure (preferably 175 MPa or greater) using a high-pressure emulsifying apparatus to obtain the oil-in-water emulsified cosmetic.

### Packaging

The perfume composition may be packaged in a container, possibly with an application means. The application means may be a spray means or a ball.

The invention also relates to a bottle provided with an application means and a packaging means, said packaging means containing the oil-in-water emulsified perfume cosmetic as described above.

The application means may be an aerosol or a hand pump or a ball. The composition may be applied in the form of fine droplets by means of pressurization devices. These devices are well known to the skilled person and include non-aerosol pumps or “atomisers”, aerosol containers comprising a propellant as well as aerosol pumps using compressed air as propellant.

The perfume composition of the invention may be an eau de toilette, an eau de Cologne, a perfume extract, a perfume spirit, a perfume veil, a water perfume or an eau de parfum.

“Perfume cosmetic” or “perfume” means a product in liquid form intended to perfume an individual after spraying or applying it to the skin, hair or clothing. Such a product is not rinsed after application. A perfuming composition will thus be distinguished from a perfumed composition. A cosmetic composition can be perfumed without being fragrant.

### Cosmetic Method

It is a further object of the invention to provide a cosmetic perfuming method comprising a step of applying to the keratinous materials or to clothing of a person, the oil-in-water emulsified perfume cosmetic as described above.

This method may be a method for perfuming the skin or hair of an individual, which comprises applying, to the skin or to hair of the individual, the composition described above. The composition may also be applied to clothing; however, it is preferred to be applied directly to the skin, preferably to a part of the body other than the face.

The perfume cosmetic is preferably applied by means of a spray means.

The invention will now be illustrated by examples, with the understanding that the invention is not meant to be limited to these examples.

### EXAMPLES

A perfuming cosmetic according to the invention and a comparative perfuming cosmetic have been prepared. The contents (mass %) of each of the materials are shown in Table 1. Comparative example 1 is identical to Example 1 except that it contains no high gravity oil.

TABLE 1
Formula

| | | | | Comparative |
|---|---|---|---|---|
| | | | Example 1 | Example 1 |
| | | INCI/COMMERCIAL NAME | mass % | mass % |
| Aqueous | A | AQUA | Qsp 100 | Qsp 100 |
| Phase | A | BUFFER AND | 1.57 | 1.57 |
| | | PRESERVATIVES | | |
| | A | PENTYLENE GLYCOL | 3 | 3 |
| | A | POLOXAMER 407 | 0.2 | 0.2 |
| | | (SYNPERONIC PE/F127-FL) | | |
| Emulsifier | B | GLYCERIN | 10 | 10 |
| phase | B | BUTYLENE GLYCOL | 10 | 10 |
| | B | HYDROGENATED | 1 | 1 |
| | | LECITHIN | | |
| | | (PHYTOCOMPO-PP ®) | | |
| Oil Phase | C | PERFUME | 12 | 12 |
| | C | CAPRYLIC/CAPRIC/ | 3 | 0 |
| | | SUCCINIC TRIGLYCERIDE | | |
| | | (MIGLYOL ® 829 MB) | | |
| Total | | | 100 | 100 |
| amount | | | | |
| Stability | | Stability 1 month at 25° C. | Yes | No |
| evaluation | | Stability 1 month at 50° C. | Yes | No |

### <Process for Preparing the Perfume Cosmetics>

The oil-in-water emulsified cosmetics were prepared by the following method. First, the aqueous phase was prepared by heating the ingredients, and then mixing and stirring at 2500 rpm using a stirrer. Second, the emulsifier phase was prepared in the same conditions as for the water phase. The oil phase was prepared at room temperature and homogenized. The emulsifier phase and the oil phase were mixed before adding water phase. Once both phases were well mixed, the water phase was added and all phases were mixed. After cooling the mixture to room temperature, a high-pressure homogenizer (Star Burst®: product of Sugino Machine, Ltd.) was used at a pressure of 140 to 200 MPa to obtain the oil-in-water emulsified perfume cosmetic.

### <Evaluation of Stability>

The cosmetic of Example 1 prepared above was poured in two transparent containers and sealed with a lid. One container was stored at 25° C. for one month, or the other container was stored at 50° C. for one month.

The same protocol was repeated with Comparative Example 1.

After storage of the containers, the absence or presence of separation between the oil phase and the water phase was observed. When no separation between an oil phase and a water phase had been observed both at 50° C. and at 25° C. was evaluated “Yes”; and that for which a separation between an oil phase and a water phase had been observed either at 50° C. or at 25° C. was evaluated “No.”

This comparative nanoemulsion was not stable.

### <Measurement of Average Particle Size>

The average particle size of the cosmetics was measured using a particle size measuring device (DelsaMax® CORE: manufactured by Beckman Coulter, Inc.) according to dynamic light scattering. The average particle size of the particles was between 100 nm and 300 nm.

1. An oil-in-water emulsified perfume cosmetic comprising less than 10% of a lower alkyl alcohol, a) at least 40% of water, b) at least 6% of a perfume concentrate, c) at least one hydrogenated lecithin, and d) at least one high gravity oil having a density at 20° C. of at least 0.90 g/cm³, the percentages being by mass relative to the mass of the oil-in-water emulsified perfume cosmetic.

2. The oil-in-water emulsified perfume cosmetic according to claim 1, comprising at least 50% of water.

3. The oil-in-water emulsified perfume cosmetic according to claim 1, wherein the perfume concentrate amount ranges from 6% to 40%.

4. The oil-in-water emulsified perfume cosmetic according to claim 1, wherein the hydrogenated lecithin amount ranges from 0.1% to 5.0%.

5. The oil-in-water emulsified perfume cosmetic according to claim 1, wherein the at least one high gravity oil has a density at 20° C. that is from 0.95 g/cm³ to 1.10 g/cm³.

6. The oil-in-water emulsified perfume cosmetic according to claim 1, wherein the at least one high gravity oil is a caprylic/capric/succinic triglyceride oil.

7. The oil-in-water emulsified perfume cosmetic according to claim 1, wherein the at least one high gravity oil amount ranges from 0.1% to 5.0%.

8. The oil-in-water emulsified perfume cosmetic according to claim 1, wherein the lower alkyl alcohol content is less than 5.0%.

9. The oil-in-water emulsified perfume cosmetic according to claim 1, being free of ethanol.

10. The oil-in-water emulsified perfume cosmetic according to claim 1, further comprising from 10.0% to 20.0% of at least one polyol comprising from 2 to 6 carbon atoms and from 2 to 4 hydroxyl groups.

11. The oil-in-water emulsified perfume cosmetic according to claim 1, comprising a mixture of three polyols being a mixture of glycerin, pentylene glycol and butylene glycol.

12. A process for preparing the oil-in-water emulsified perfume cosmetic according to claim 1, comprising a step of emulsifying a mixture of components (a) to (d) in a high-pressure homogenizer at a pressure being 100 MPa or higher.

13. A bottle provided with an application means and a packaging means, said packaging means containing the oil-in-water emulsified perfume cosmetic according to claim 1.

14. A cosmetic perfuming method comprising a step of applying to the keratinous materials or to clothing of a person an oil-in-water emulsified perfume cosmetic according to claim 1.

15. The oil-in-water emulsified perfume cosmetic according to claim 1, wherein the lower alkyl alcohol is ethanol.

16. The oil-in-water emulsified perfume cosmetic according to claim 1, comprising at least 60% of water.

17. The oil-in-water emulsified perfume cosmetic according to claim 1, wherein the perfume concentrate amount ranges from 10% to 30%.

18. The oil-in-water emulsified perfume cosmetic according to claim 1, wherein the hydrogenated lecithin amount ranges from 0.3% to 1.0%.

19. The oil-in-water emulsified perfume cosmetic according to claim 1, wherein the at least one high gravity oil amount ranges from 0.5% to 2.0%.
